(19) United States
(12) Patent Application Publication
(10) Pub. No.: US 2008/0226701 A1
(43) Pub. Date: Sep. 18, 2008

(54) MEDICATED PATCHES

(76) Inventors: John Deignan, Massapequa Park, NY (US); Donald Giambolvo, Dix Hills, NY (US)

Correspondence Address: KATTEN MUCHIN ROSENMAN LLP, 575 MADISON AVENUE, NEW YORK, NY, 10022-2585, US

(21) Appl. No.: 12/045,293

(22) Filed: Mar. 10, 2008

Related U.S. Application Data
(60) Provisional application No. 60/918,106, filed on Mar. 15, 2007.

(51) Int. Cl. A61F 13/12; A61K 9/00

(52) U.S. Cl. 424/449; 424/400

(57) ABSTRACT

An athletic head gear having a hard outer shell, at least one soft inner padding cushioning the head of a user from the hard outer shell, and a medicated patch including an adhesive layer contacting the soft inner padding, and a medicated layer for contacting the user's skin, where the medicated patch includes a medicament useful for treating or preventing acne.

### BACKGROUND OF THE INVENTION

1. Field of the Invention

The present invention is directed to medicated patches and particularly to medicated patches contacting the skin of a user for the treatment and prevention of acne.

2. Description of Related art

Unwanted skin conditions such as common acne are a constant problem with many individuals and are a major treatment concern of many dermatologists. It is estimated that as many as 32 million Americans exhibit some form of unwanted acne.

The treatment of acne is of major concern to dermatologists. Acne accounts for more than four million visits to dermatologists each year. Typically, acne arises in the early teen years and subsides by the mid twenties. In many cases, particularly in women, acne remains a chronic problem well into the adult years. It is estimated that as many as 32 million Americans suffer from acne.

One of the most common forms of acne is the result of the secretion of sebum by the sebaceous gland into a blocked pore. Continued secretion results in buildup of the sebum in the blocked pore. Bacteria in the pore cause infection and a common unsightly skin condition known as pimples. Sebaceous gland hyperplasia is also a common form of acne in which the sebaceous gland grows or become enlarged as a result of overproduction of sebum. A pimple is formed even if the gland is not blocked.

To combat the formation of acne, one of the most common approaches is to regularly wash the skin of the affected area in an effort to prevent the blockage of pores and to remove dirt and oils from the skin which and cause the pore blockages.

In addition, there are a variety of topical treatments that can be applied to the skin and are generally used to remove oils from the skin in the affected area. One a result of the drying properties the creams and other topical treatments is that they tend to be prone to unsightly caking on the portion of the skin on which they are applied. This caking has several disadvantages. First it is unsightly, which is a major concern for most people affected with acne. Second, the treatments often interfere with or directly contrast with make-up used by women and girls in order to improve their appearance. Third, the caked on topical treatments tends to crack and fall away from the applied area resulting in less than desired treatment and considerable waste of medicament.

For example, one common technique is to apply these topical treatments before going to bed. As a result, once dried, the topical treatments are often rubbed off while the user is sleeping, thus limiting their effectiveness and creating a mess to be cleaned up.

Another problem with the topical treatments is that when engaged in physical activity these treatments rub of and fail to be effective. Unfortunately, this is exactly the time when a user might most benefit from treatment options. While engaging in physical activities, particularly those where the participant wears some sort of headgear, the sweat and oils produced sit on the skin, are often wiped off by hand or on a sweaty shirt, with the result that the incidence of acne increases. Even further, as mentioned, the head gear such as helmets and the like further compounds the problem by actually engraining the dirt and oils into the skin, and ensuring that there is continued contact with the skin.

Accordingly, there is a need for a method and device for treating acne that can address the shortcomings of the known acne treatment methods.

### SUMMARY OF THE INVENTION

One aspect of the present invention is directed to an athletic head gear having a hard outer shell, at least one soft inner padding cushioning the head of a user from the hard outer shell, and a medicated patch including an adhesive layer contacting the soft inner padding, and a medicated layer for contacting the user's skin, where the medicated patch includes a medicament useful for treating or preventing acne.

Another aspect of the present invention is directed to an acne treatment device including a backing layer, and an adhesive/medicated layer, wherein said treatment device is applied to a user's skin for the treatment or prevention of acne, and the combination of the backing layer and adhesive/medicated layer forms a fanciful shape appealing to the user.

### BRIEF DESCRIPTION OF THE DRAWINGS

FIG. 1 is a cross sectional view of a medicated patch according to the present invention;

FIG. 2 is a bottom view of a baseball batting helmet according to one aspect of the invention;

FIG. 3 is a front view of the baseball batting helmet of FIG. 2;

FIG. 4 is a profile view of a user wearing a medicated patch according to one aspect of the present invention;

FIG. 5 is a cross sectional view of a medicated patch according to a second embodiment of the present invention;

FIG. 6 is a photograph of a football chin strap showing a further embodiment of the present invention;

FIG. 7 is a photograph of a football helmet showing a further aspect of the present invention;

FIG. 8 is a photograph of a football helmet showing a further aspect of the present invention;

FIG. 9 is a photograph of a football helmet showing a further aspect of the present invention;

FIG. 10 is a photograph of a football helmet showing a further aspect of the present invention;

FIG. 11 is a photograph of a hat showing a further aspect of the present invention; and

FIG. 12 is a photograph of a hat showing a further aspect of the present invention.

Other features and advantages of certain embodiments of the present invention will become more fully apparent from the following description taken in conjunction with the accompanying figures and claims.

### DETAILED DESCRIPTION

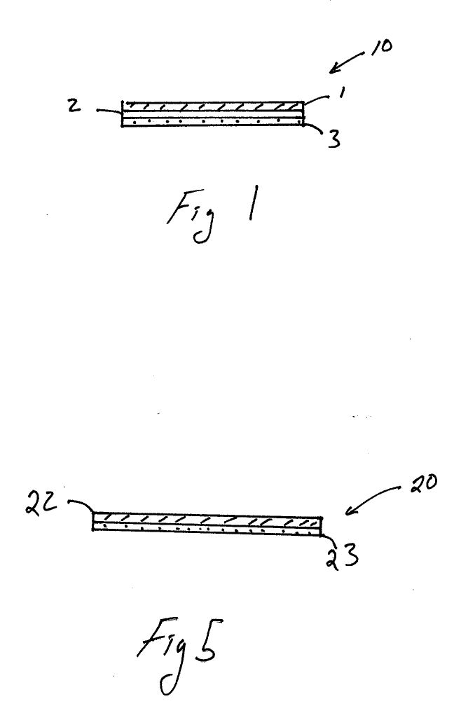

A first aspect of the present invention can be seen in FIG. 1 showing a medicated patch 10, composed of an adhesive layer 1, a structural layer 2, and a medicated layer 3. In one embodiment, the medicated layer 3 is formed such that upon application of pressure to the pad by for example, the user's head a small amount of anti-acne medication is released. The adhesive layer 1 and the medicated layer 3 may include peel away cellophane, thus making storage of the strips easier. These patches are easily placed into positions and easily removed for replacement with new pads after each use of the helmet, or at least on a daily basis.

The medicated layer 1 is impregnated with preventative medicine that can be either time release or released by perspiration produced by wearer. The patches bathe the areas of skin they contact prevent the growth of bacteria that causes acne and also cleanses the skin of oils and dirt that are promoters of acne. This release can be either a rapid release to instantly cover the affected area, or a slow release, wherein the user is exposed to more medication the longer the helmet is worn.

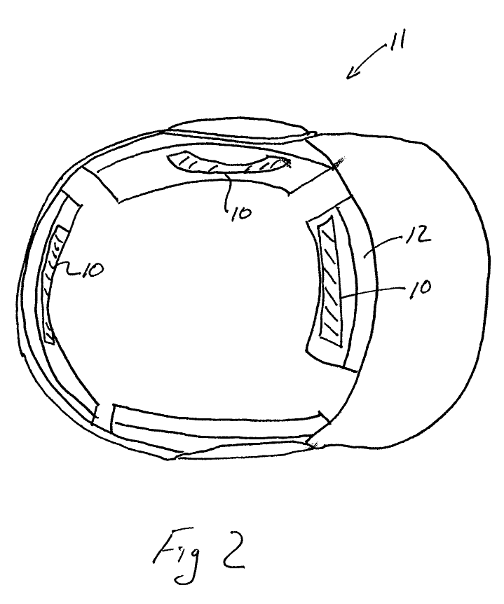

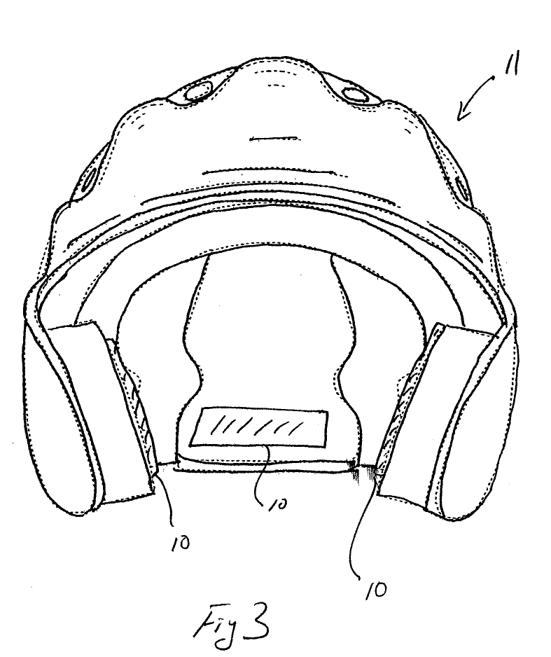

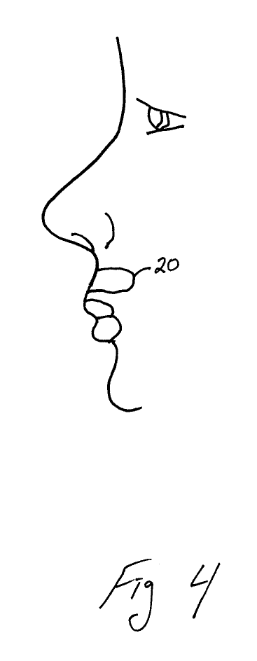

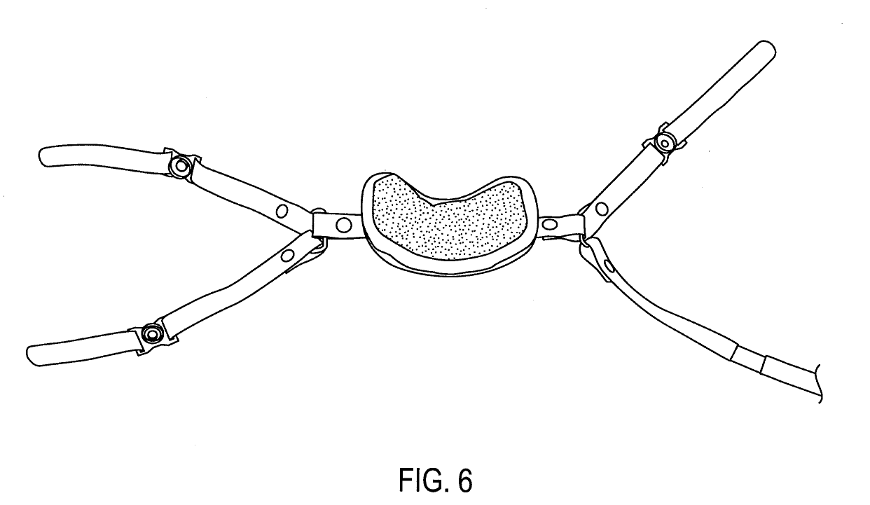

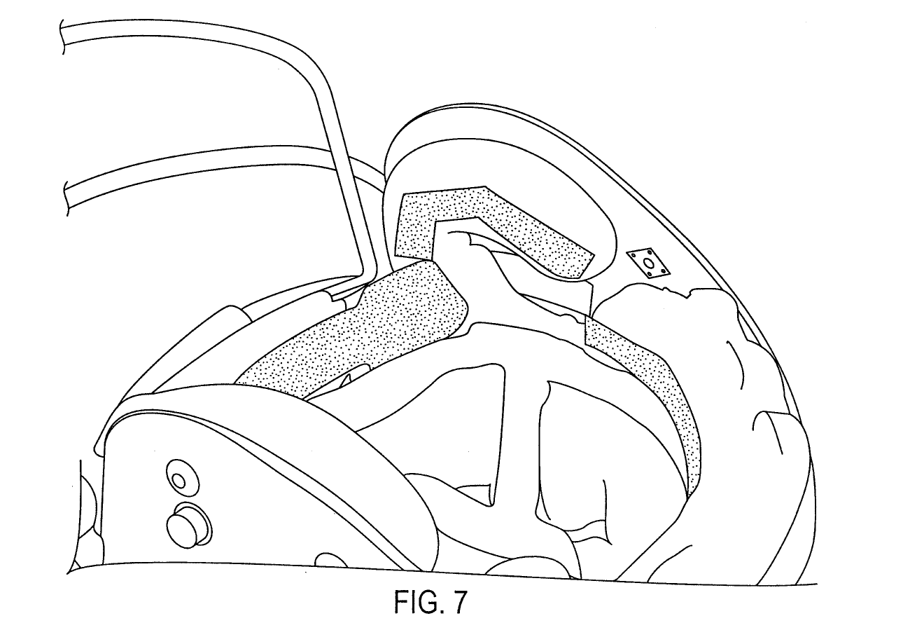

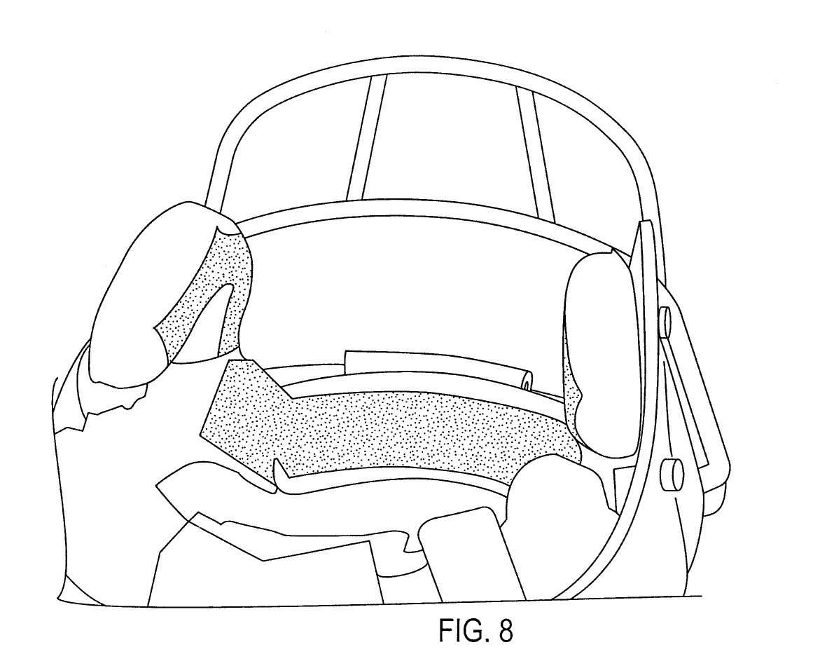

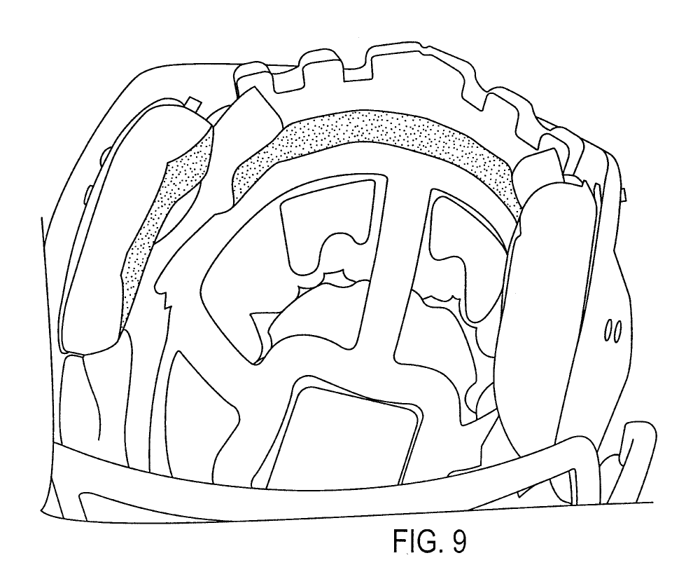

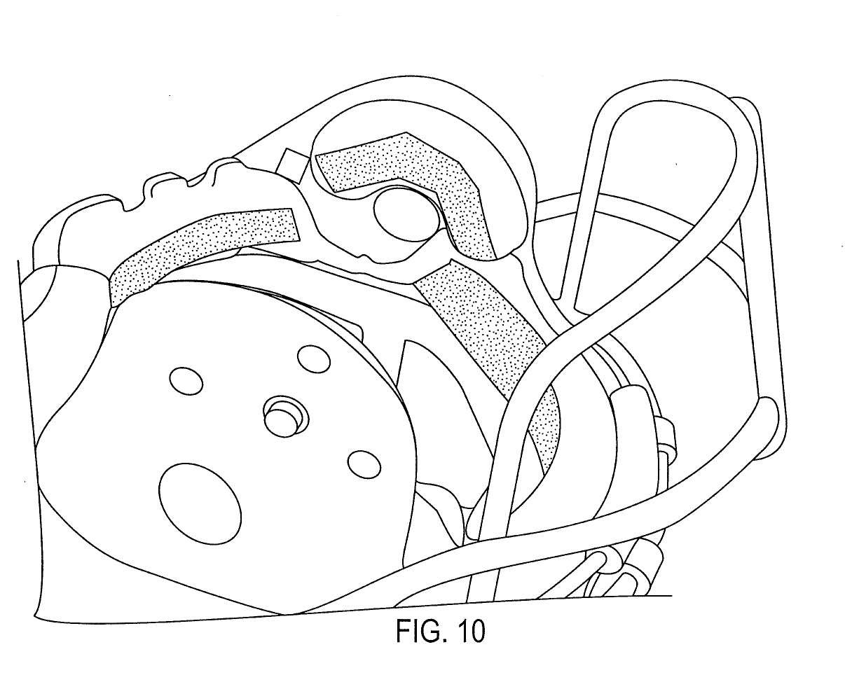

The medicated pad can be shaped to fit into any of a variety of locations for use with protective helmets and the like. The adhesive layer 3 allows the patches have to be inserted in and placed in helmets on the point of contact with the skin. For example, in the baseball helmet 11 of FIG. 2, the medicated pad can be fit to rest on the inside of the helmet 11, where a foam pad 12 protects the forehead of the user from shock, as shown in FIG. 2. Another convenient location is around the top of the ear, which forms a saddle between the ear and the head of a user and collects dirt and oil and is prone to acne. The location of a medicated patch 10 over the ear is also shown in FIG. 2. Other sizes and shapes that are contemplated for the medicated patch include to conform to a chin strap (FIG. 6), cheek/ear pads, (FIGS. 7-10) and patches to conform to the back of the neck on the helmet (FIGS. 9-10), those of skill in the will appreciate that other shapes are also contemplated within the scope of this invention.

According, to another aspect of the invention, the pads of the helmet itself can be constructed to be replaceable and have impregnated within themselves the anti-acne or acne treatment medication.

A further aspect of the invention is that the medicated pads can also be applied to other types of sports equipment which is worn close to the body. Such equipment includes for example, shoulder pads for football and lacrosse, elbow pads, knee pads, knee braces, ski helmets, ski masks, wrestling head gear, motorcycle helmets, bicycle helmets, face shields, ski goggles, woolen caps, scarves, eyeglass nose pads, and others. Indeed, the medicated pads, may be applied to any article which is worn close to the body and can lead to the formation of acne.

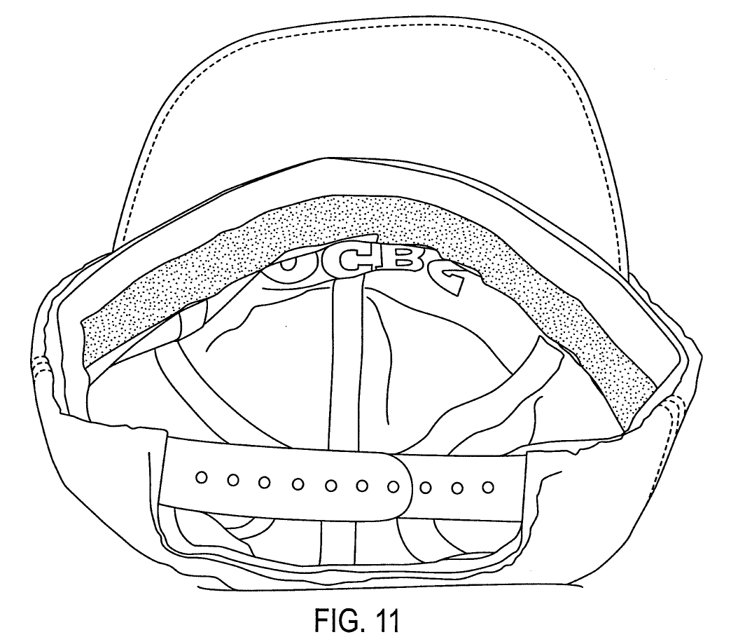

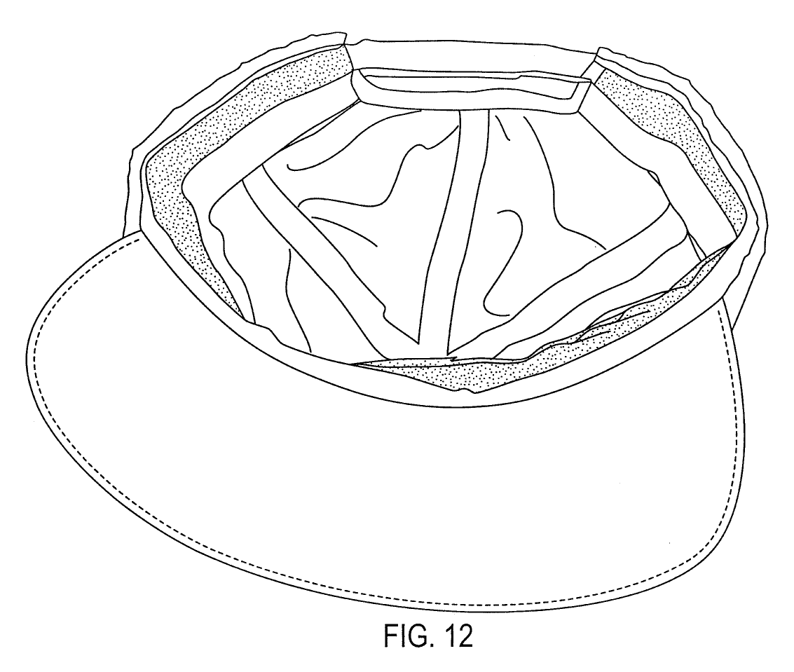

For example, another possible use includes on the inside of baseball types hats as shown in FIGS. 11 and 12. As shown there a user can apply the medicated patch 10 to the internal head band of the hat and prevent or treat acne.

Still a further aspect of the present invention is a headband as commonly worn by those who play tennis, soccer, and basketball to prevent the dripping of sweat into the eyes and face of a wearer. In this embodiment the headband may be disposable and intended to be donned each time the user participates in the activity. The headband is impregnated with the anti-acne or acne treatment medication and operates in a similar fashion to those devices discussed above. Alternatively, the medicated pads 10 discussed above, can be applied to a traditional head band Besides a preventive medication formula, the medicated patches 10 may alternatively include a medicinal formula that treats active acne also.

The medicated patches 10 to be adhered to the inside of helmets at the forehead and back of neck strips may be approximately 1.5″ to 2″ wide×12″ long with a perforated stripe every inch or two for easy breakaway in order to customize the length of strip to fit different size helmets.

Particularly for relatively strait run areas such as for the forehead and the back of the neck, the medicated patches may be dispensed as strips placed on a roll, with a dispenser, where a user can pull out the length needed as one would a roll of tape. This roll of medicated patches may include perforations at regular intervals, or alternatively, simply be cut with scissors for application.

Another aspect of the present invention is that, in order for the medicated patches to adhere to helmet pads they will need a strong adhesive so when the helmets are put on and taken off the med. strips do not fall off. As a practical matter, strong adhesives may damage the helmet pads when the medicated patches are removed and replaced. To combat this, the present invention contemplates the use of an additional cellophane tape that the user attaches to the helmet pads semi-permanently. This tape (not shown) has two sides. One side is a strong adhesive on it to stick to the helmet pads. The other side has a coarse texture so that the medicated patches can adhere to the tape and not fall off. The medicated patches can then be applied to the tape and easily be removed and changed without damaging the helmet pads. The tape will remain in the helmet until it starts to wear out. The tape can then easily be removed with alcohol swabs and then be replaced with a new piece of tape.

A second embodiment of the present invention is directed to medicated patches 20, which treat and/or prevent acne much in the way other topical treatments, but where as topical treatments often dry out and crack, or are otherwise unsightly, the patches of the present invention remain intact.

According to this second embodiment, as shown in FIG. 5, the medicated patches 20 include a medication/adhesive layer 23 which is placed against the skin of a user, and a backing and structural layer 22 for rigidity of the patch 20. Like other topical treatments, the patches 20 are placed on the skin in areas that are prone to acne and left there during their effective period.

The patches 20 may be formed in different sizes to accommodate chins, cheeks, foreheads, etc.

Over the medicated adhesive layer 23, a peel away cellophane may be placed on the patch for ease of storage and transport. The medicated adhesive layer 23 is impregnated with acne preventative medication that may be time released and bathe the areas of face and skin that are prone to acne. The adhesive/medicated layer may be for example a cellophane tape.

The two layers 22 and 23 together may be clear, be pigmented so that the medicated patch 20 takes on a skin tone or preferably shaped and formed so that they take on designer looks with designers colors.

Because of childhood vanity it is believed that there may be interest in designer looks and colors, particularly those directed to for example, NFL teams, Baseball teams, Disney figures, Skull and Cross Bones, etc. or any other acceptable design that young adults would not be embarrassed to wear in public. Indeed all of the possibilities available to fake tattoos, which have strong following with adolescents and teens are available to the medicated patches 20, with the added bonus that they are treating and preventing acne. The medicated patches be changed many different times a day to adjust to clothing, makeup or any other reason that makes the child or adult happy and comfortable. Further, these patches have the added benefit of not looking or acting like of creams that crack while wearing them. Moreover, it has been experienced that such patches may be preferable to young men who don't like the creams because they don't to appear as if they are wearing makeup and to women and girls who don't like the way creams clash or interfere with their makeup

A third embodiment of the present invention is directed to a night time acne prevention and treatment kit. The kit includes a facial cleanser liquid preparation that will then be used with medicated patches that are adhered to the body. The medicated patches include medication as discussed above. These pads are worn preferably at night while sleeping to combat acne that is present. The pads come in a variety of shapes and sizes for different sized individuals and to be applied to specific areas of the body. Unlike other topical treatment methods such as creams, the patches containing the medicine are less prone to rubbing of on pillows and sheets. These patches may have all the features of the patches described above in connection with the second embodiment.

I claim:

1. An athletic head gear comprising:
a hard outer shell;
at least one soft inner padding cushioning the head of a user from the hard outer shell; and
a medicated patch including an adhesive layer contacting the soft inner padding, and a medicated layer contacting the user's skin, wherein the medicated patch includes a medicament useful for treating or preventing acne.

2. The athletic head gear of claim 1, wherein the medicament is a slow releasing agent.

3. The athletic head gear of claim 1, wherein the medicament is a rapid release agent.

4. The athletic head gear of claim 1, wherein the medicament is released by the user's perspiration.

5. The athletic head gear of claim 1, wherein the adhesive layer of the medicated patch is adhered to a cellophane tape which is itself adhered to the soft inner padding.

6. An athletic head gear comprising:
a hard outer shell;
at least one soft inner padding cushioning the head of a user from the hard outer shell, wherein said inner padding includes a medicated layer contacting the user's skin, wherein the medicated includes a medicament useful for treating or preventing acne.

7. The athletic head gear of claim 6, wherein the medicament is a slow releasing agent.

8. The athletic head gear of claim 6, wherein the medicament is a rapid release agent.

9. The athletic head gear of claim 6, wherein the medicament is released by the user's perspiration.

10. An acne treatment device comprising:
a backing layer; and
an adhesive/medicated layer, wherein said treatment device is applied to a user's skin for the treatment or prevention of acne, and the combination of the backing layer and adhesive/medicated layer forms a fanciful shape appealing to the user.

11. The acne treatment device of claim 10, wherein the medicament is a slow releasing agent.

12. The acne treatment device of claim 10, wherein the medicament is a rapid release agent.

13. The acne treatment device of claim 10, wherein the medicament is released by the user's perspiration.

14. The acne treatment device of claim 10, wherein the device is formed into a long strip from which a desired amount is removed for use.

15. The acne treatment device of claim 10, wherein the backing layer includes a fanciful design.

16. An acne treatment device comprising:
an adhesive layer; and
a medicated layer, wherein said treatment device is applied to an article of equipment worn by a user for the treatment or prevention of acne.

17. The acne treatment device of claim 16, wherein the device is applied to an article selected from the group consisting of shoulder pads , elbow pads, knee pads, knee braces, ski helmets, ski masks, wrestling head gear, motorcycle helmets, bicycle helmets, face shields, ski goggles, woolen caps, scarves, and eyeglass nose pads.

18. The acne treatment device of claim 16, wherein the device is formed into a long strip from which a desired amount is removed for use.

19. The acne treatment device of claim 16, wherein a medicament in the medicated layer is a slow releasing agent.

20. The acne treatment device of claim 16, wherein a medicament in the medicated layer is a rapid release agent.

21. The acne treatment device of claim 16, wherein a medicament in the medicated layer is released by the user's perspiration.
